(19) United States
(12) Patent Application Publication
(10) Pub. No.: US 2026/0085335 A1
(43) Pub. Date: Mar. 26, 2026

(54) PROVIDING AN OIL COMPOSITION THROUGH FERMENTATION OF BIOMASS WITH A YEAST

(71) Applicant: NOPALM INGREDIENTS B.V., Wageningen (NL)

(72) Inventors: Jeroen HUGENHOLTZ, Wageningen (NL); Rudolf Marcel VELOO, Wageningen (NL); Kirsten Cornelia Adriana DEKKERS, Wageningen (NL); Maria TWIGT, Wageningen (NL)

(21) Appl. No.: 19/108,821

(22) PCT Filed: Aug. 29, 2023
(86) PCT No.: PCT/NL2023/050441

§ 371 Date: Mar. 5, 2025

(30) Foreign Application Priority Data
Sep. 5, 2022 (NL) 2032949

(51) Int. Cl. C12P 7/6463; C11B 3/16; C12N 1/06; C12N 1/16; C12R 1/01

(52) U.S. Cl. CPC C12P 7/6463; C11B 3/16; C12N 1/063; C12N 1/066; C12N 1/16; C12R 2001/01

(57) ABSTRACT

The invention provides a method for producing an oil composition (100) from biomass (200), wherein the method comprises: a preparation stage (11) comprising providing a growth medium (300), wherein the growth medium (300) comprises the biomass (200), wherein the growth medium (300) comprises 1-10 wt % dry matter, 0.02-30 wt % of a carbon source, 0.01-5 wt % of a nitrogen source, 0.00001-0.05 wt. % of a magnesium source, and wherein the growth medium (300) has a pH selected from the range of 3.0-8.0, wherein the biomass (200) comprises a non-pasteurized biomass (220); a fermentation stage (12) comprising (a) inoculating the growth medium (300) with a yeast cell (400), thereby providing an cell suspension (500), and (b) controlling a temperature of the cell suspension (500) in the range of 10-40° C., wherein the yeast cell (400) is selected from the species Yarrowia lipolytica, and Cutaneotrichosporon oleaginous, and wherein the fermentation stage is continued until the cell suspension (500) reaches a cell density selected from the range of 1E8-1E10 cells/ml, wherein the pH of the cell suspension (500) is not controlled during the fermentation stage (12); an oil procurement stage (15) comprising separating a yeast oil (110) from the cell suspension (500) to provide the oil composition (100), wherein the oil procurement stage comprises mechanically lysing the cell suspension (500) to provide a lysed cell suspension (600), wherein the mechanical lysing (610) comprises one or more of screw-pressing (630), bead beating, French pressing and homogenizing.

### FIELD OF THE INVENTION

The invention relates to a method for producing an oil composition from biomass. The invention further relates to a process for the production of a product comprising an oil composition. The invention additionally relates to a product comprising an oil composition.

### BACKGROUND OF THE INVENTION

Methods for producing an oil composition from biomass are known in the art. For instance, AU2010236412A1 relates to methods of microbial oil extraction and separation, wherein lipids can be extracted from a microbial biomass that constitutes at least 20% lipids by weight and has a moisture content of less than 4% by weight by applying pressure to the biomass so as to release lipids therefrom, thereby leaving a biomass of reduced lipid content; and collecting the lipids.

WO2011130576A1 describes novel oleaginous yeast biomass, yeast oil, food compositions comprising oleaginous yeast biomass, whole oleaginous yeast cells, and/or yeast oil in combination with one or more edible ingredients, and methods of making such compositions by combining oleaginous yeast biomass or yeast oil with other edible ingredients.

WO2010120939A2 describes lipids that can be extracted from a microbial biomass that constitutes at least 20% lipids by weight and has a moisture content of less than 4% by weight by applying pressure to the biomass so as to release lipids therefrom, thereby leaving a biomass of reduced lipid content; and collecting the lipids.

WO2011130573A1 describes oleaginous yeast that can be used to produce oil that can be extracted from oleaginous yeast biomass and converted into a wide variety of useful products, including fuels, hydrocarbon compositions, and/or oleochemicals.

WO2012061647A2 describes methods and compositions for the production of dielectric fluids from lipids produced by microorganisms are provided, including oil-bearing microorganisms and methods of low cost cultivation of such microorganisms. Microalgal cells containing exogenous genes encoding, for example, a sucrose transporter, a sucrose invertase, a fructokinase, a polysaccharide-degrading enzyme, a lipid pathway modification enzyme, a fatty acyl-ACP thioesterase, a desaturase, a fatty acyl-CoA/aldehyde reductase, and/or an acyl carrier protein are useful in manufacturing dielectric fluids.

### SUMMARY OF THE INVENTION

Vegetable oil compositions are widely used in food manufacturing, cosmetic products, or as a biofuel due to availability, relatively low costs, safety for human consumption and exposure, and consistent quality in processed goods. However, the production of vegetable oil compositions, such as especially palm oil, but also soy oil and coconut oil, may have been associated with environmental and humanitarian concerns, resulting in industrial and consumer demand for more ethical and sustainable alternative oil compositions, which may preferably mimic the oil composition they intend to replace, such as palm oil, and which may preferably rely on natural processes.

It may be challenging to replace abovementioned vegetable oils by other plant oils as plants may have a relatively stable oil composition, i.e., plant-based processes may offer little possibilities for tuning the oil composition based on a selection for specific process parameters. The prior art may describe isolating specific individual oils and fatty acids from plant oils and subsequently recombining such oils and fatty acids to provide desired compositions. This may require extensive downstream processing, which may be energy-intensive and/or costly.

The prior art may describe processes for providing alternative oil compositions using microorganisms, such as using algae, or such as using yeasts. However, there remains a need for simpler, more efficient, and more flexible processes for providing microbial oil compositions.

For instance, prior art approaches may generally require complex setups, wherein microbial growth requires well-defined media, sterilized equipment, continuous monitoring, and wherein process parameters, such as pH, oxygen, and nutrient availability, are (automatically) controlled throughout the process. Besides complex, such setups may also be relatively expensive and/or energy-intensive.

Further, prior art approaches may be inflexible in that they relate to the use of a specific feedstock, such as a specific biomass source, and/or in that they relate to providing a specific oil composition.

It may be preferable that the produced oil composition(s) can directly replace vegetable oil compositions, such as palm oil, coconut oil, and soy oil, in their typical applications. However, prior art microbial oils may substantially differ from vegetable oil compositions, which may complicate their use in established processes and products.

Similarly, it may be preferable that the produced oil composition(s) are produced without genetically modified organisms (GMOs), both in view of (local) legislation and consumer demands.

Further, the prior art may describe extracting oil from yeast cells using solvents. Such extraction approaches may typically involve the use of relatively large amounts of environmentally unfriendly solvents such as hexane and chloroform.

Hence, it is an aspect of the invention to provide an alternative method for producing an oil composition from biomass, which preferably further at least partly obviates one or more of above-described drawbacks. The present invention may have as object to overcome or ameliorate at least one of the disadvantages of the prior art, or to provide a useful alternative.

In a first aspect, the invention may provide a method for producing an oil composition from biomass. In embodiments, the method may comprise one or more of a preparation stage, a fermentation stage, and an oil procurement stage. The preparation stage may comprise providing a growth medium (or: “fermentation medium”), especially an initial growth medium. The growth medium may comprise the biomass. The growth medium may further comprise one or more of 1-10 wt % dry matter, 0.02-30 wt % of a first carbon source, 0.01-5 wt % of a first nitrogen source, and 0.00001-0.05 wt % of a magnesium source. Especially, the growth medium may have a pH selected from the range of 3.0-8.0. In embodiments, the fermentation stage may comprise inoculating the growth medium with a yeast cell, especially wherein the yeast cell is selected from the species Yarrowia lipolytica or Cutaneotrichosporon oleaginous, especially from the species Y. lipolyitica. Thereby, a cell suspension (or: “yeast suspension”) may be provided. Further, the fermentation stage may comprise controlling a temperature of the cell suspension in the range of 10-40° C. The fermentation stage may especially be continued until the cell suspension reaches a (yeast) cell density selected from the range of 1E8-1E10, especially from the range of 1E9-1E10. In embodiments, the oil procurement stage may comprising separating a yeast oil from the cell suspension to provide the oil composition. This may be achieved via mechanically lysing the cell suspension, especially thereby providing a lysed cell suspension. The mechanical lysing may, in embodiments, comprise one or more of screw-pressing, bead beating, French pressing and homogenizing.

Therefore, in specific embodiments the invention provides a method for producing an oil composition from biomass, wherein the method comprises: a preparation stage comprising providing a growth medium, wherein the growth medium comprises the biomass, wherein the growth medium comprises 1-10 wt % dry matter, 0.02-30 wt % of a first carbon source, 0.01-5 wt % of a first nitrogen source, 0.00001-0.05 wt. % of a magnesium source, and wherein the growth medium has a pH selected from the range of 3.0-8.0; a fermentation stage comprising (a) inoculating the growth medium with a yeast cell, thereby providing a cell suspension, and (b) controlling a temperature of the cell suspension in the range of 10-40° C., wherein the yeast cell is selected from the species Yarrowia lipolytica, and wherein the fermentation stage is continued until the cell suspension reaches a cell density selected from the range of 1E8-1E10; an oil procurement stage comprising separating a yeast oil from the cell suspension to provide the oil composition via mechanically lysing the cell suspension to provide a lysed cell suspension, wherein the mechanical lysing comprises one or more of screw-pressing, bead beating, French pressing and homogenizing.

With the present invention, the method for producing an oil composition from biomass may provide an alternative method to provide a oil composition with characteristics such that it may replace currently available vegetable oil compositions, especially those associated with environmental and humanitarian concerns. The current method provides a method that may require no specialized fermentation equipment and may be applicable to a wide variety of biomass sources, hence it may be an easily available and economically feasible method to provide safe and ethical oil compositions with consistent desired characteristics.

Further, the method of the invention may facilitate including (desirable) compounds deriving from the biomass and/or yeast cell in the oil composition, such as flavor compounds (citrus, herbs), fat-soluble antioxidants, and/or fat-soluble vitamins.

As indicated, the invention may provide a method for producing an oil composition from biomass, the method comprising one or more stages. The first stage may be the preparation stage. The preparation stage may especially comprise providing a growth medium with a composition and characteristics making it suitable for oleaginous fermentation. The preparation stage may then be followed by the fermentation stage. In the fermentation stage, the growth medium may be inoculated with a yeast cell to provide a cell suspension. The yeast cell may grow and produce yeast oil during the fermentation stage until a predefined amount of yeast cells (or “yeast biomass”) are (“is”) present in the cell suspension. The fermentation stage may be followed by the oil procurement stage. During the oil procurement stage, the yeast cell in the cell suspension is mechanically lysed such that the yeast oil is released from the yeast cell. Following this step, the oil composition may be obtained by separating the yeast oil from other components in the lysed cell suspension. The term “stage” and similar terms used herein may refer to a (time) period (also “phase”) of a method and/or an operational mode. The different stages may (partially) overlap (in time). For example, the preparation stage may, in general, be initiated prior to the fermentation stage, but may partially overlap in time therewith. However, for example, the fermentation stage may typically be completed prior to the oil procurement stage. It will be clear to the person skilled in the art how the stages may be beneficially arranged in time.

The selection of growth medium conditions, yeast cell, and mechanical lysing method of the invention may provide a relatively simple process for providing an (alternative) oil composition, while the process is flexible in terms of compatible biomass sources, as well as flexible in terms of the target oil composition. In particular, the growth medium may be selected such that essentially no nutrients need to be added during the fermentation stage, especially during a nutrient abundance stage (see below), and that a well-timed transition from yeast growth to oil production naturally occurs. Further, by setting (or controlling) a temperature during the fermentation stage, the fatty acid profile of the oil composition may be beneficially steered towards a target oil composition.

The method of the invention may especially comprise one or more of a preparation stage, a fermentation stage, and an oil procurement stage.

In embodiments, the method may comprise the preparation stage. The preparation stage may especially comprise providing a growth medium. The term “growth medium” may herein especially refer to a medium providing the nutrient sources facilitating the yeast cell to grow and to produce yeast oil during the fermentation stage. The preparation stage may comprise providing the growth medium such that a suitable balance is struck between cell growth and oil production. In particular, there may be a trade-off between growth of the yeast cells and yeast oil production as both may require nutrients, such as a first carbon source. Although some yeast oil may be produced while the yeast cells are growing, the production of yeast oil may substantially increase under a specific cultivation condition, such as under nitrogen and/or magnesium nutrient limitation. In particular, the preparation stage of the invention may comprise providing a growth medium with a nutrient composition selected such that cell growth occurs until a suitable cell density is achieved, at which time a cultivation condition (naturally) is such that the yeast cells will switch to primarily producing oil rather than more yeast biomass. For instance, in embodiments, the amount of nitrogen and/or magnesium in the (initial) growth medium may be selected to facilitate growth up to a (predefined) cell density, such that after the yeast cells arrive at such cell density, they will switch to oil production, essentially without intervention during the fermentation process.

In embodiments, the growth medium may comprise one or more of biomass, a (first) carbon source, a (first) nitrogen source, and a magnesium source. The terms “carbon source”, “nitrogen source”, “magnesium source”, as well as, for instance, a “phosphor source”, a “sulfur source”, etc., may herein be collectively referred to as “nutrient sources”—i.e., a group of compounds facilitating yeast metabolism, such as for yeast cell growth, and such as for yeast oil production.

In embodiments, the growth medium may comprise biomass, especially plant(-derived) biomass. The term “biomass” may herein especially refer to a renewable organic material suitable as (part of) a feedstock for growth of the yeast cell. For instance, the biomass may comprise (partially decomposed) plant biomass (see further below). The biomass may typically comprise a variety of nutrient sources usable by the yeast cell. Hence, the biomass may comprise (or “provide”) at least part of one or more of the (first) carbon source, the (first) nitrogen source, and the magnesium source.

Hence, in embodiments, the nutrient sources (see below) in the growth medium may be at least partly be provided by the biomass, i.e., the biomass may comprise (at least part of) the nutrient sources, such as at least part of one or more of the first carbon source, the first nitrogen source, and the magnesium source.

In further embodiments, the nutrient sources in the growth medium may be entirely provided by the biomass comprised by the growth medium. In embodiments wherein the biomass as such lacks one or more nutrient sources to provide the growth medium for yeast cell growth and yeast oil production, the growth medium may be supplemented with additional nutrient sources. This may especially be the case for specific biomass sources and conditions, such as f.e. biomass obtained from the sugar refinery industry or the fruit processing industry, which may have relatively low nitrogen bioavailability, and hence may require mixing with biomass obtained from the dairy industry or the brewery industry, which may have relatively high nitrogen bioavailability.

The growth medium may comprise a first carbon source, especially wherein the first carbon source comprises one or more compounds from which the yeast cell can acquire carbon [C] for cell growth and/or oil production during the fermentation stage. The first carbon source may, in embodiments, be at least partly provided by the biomass comprised by the growth medium. Especially, the first carbon source may be entirely provided by the biomass. In other embodiments, the first carbon source may be (partially) supplemented to the growth medium. In embodiments, the first carbon source may comprise one or more bioavailable organic compounds selected from the group of glucose, maltose, ethanol, lactose, sucrose, galactose, fructose, lactic acid, acetic acid, propionic acid, butyric acid, and citric acid. The first carbon source may further serve as an energy source for the yeast cell. Hence, in embodiments, the carbon source may provide both carbon (for yeast biomass and yeast oil production) and energy.

In embodiments, the growth medium may comprise 0.02-30 wt % of the first carbon source, i.e., the amount of the first carbon source in the growth medium may be in the range of 0.02-30 wt %. In further embodiments, the amount of the first carbon source in the growth medium may be in the range of 0.05-30 wt %, such as in the range of 0.05-20 wt %. In further embodiments, the amount of the first carbon source in the growth medium may be in the range of 0.1-20 wt %, especially in the range of 0.1-15 wt %. Moreover, the amount of first carbon source in the growth medium may be in the range of 0.5-15 wt %, such as in the range of 1-15 wt %, especially in the range of 1-10 wt %.

The weight percentages of the nutrient sources mentioned herein may especially be on dry weight basis, unless specified otherwise. Hence, the phrase “the growth medium may comprise 30 wt % of a first carbon source” and similar phrases, may refer to 30 wt % of the growth medium on dry weight basis comprising the first carbon source.

The growth medium may further comprise a (first) nitrogen source, especially wherein the nitrogen source comprises one or more compounds from which the yeast cell can acquire nitrogen [N] during the fermentation stage. The nitrogen source may be at least partly provided by the biomass. Especially, the nitrogen source may be entirely provided by the biomass. In other embodiments, the nitrogen source may be (at least partially) supplemented to the growth medium.

The nitrogen source may especially comprise one or more compounds selected from the group comprising ammonium, glutamine, asparagine, urea, and amino acids.

As described above, a nitrogen limitation may be beneficial to stimulate yeast oil production during the fermentation stage. Hence, the amount of nitrogen in the growth medium may be selected to achieve a timely switch to oil generation, and thereby may facilitate achieving a particularly high oil composition yield.

Hence, in embodiments, the growth medium may comprise 0.01-5 wt % of the first nitrogen source, i.e., the amount of the nitrogen source in the growth medium may be in the range of 0.01-5 wt %. In further embodiments, the amount of the nitrogen source in the growth medium may be in the range of 0.02-5 wt %, such as 0.02-2 wt %. In further embodiments, the amount of the nitrogen source in the growth medium may be in the range of 0.05-2 wt %, especially 0.05-1 wt %. Moreover, the amount of the nitrogen source in the growth medium may be in the range of 0.1-1 wt %, such as 0.1-0.5 wt %.

In particular, the method, especially the preparation stage, may comprise providing the growth medium with an amount of the nitrogen source suitable to arrive at a target cell density (also see below) during the fermentation stage, especially to (essentially) stop growth (due to a nitrogen limitation) at a target cell density, such as of at least 5E7 cells/ml, during the fermentation stage.

The growth medium may further comprise a magnesium source, especially wherein the magnesium source comprises one or more compounds from which the yeast cell can acquire magnesium [Mg²⁺] during the fermentation stage. The magnesium source may especially comprise Mg²⁺. The magnesium source may be at least partly provided by the biomass. Especially, the magnesium source may be entirely provided by the biomass. In other embodiments, the magnesium source may be supplemented to the growth medium.

As described above, a magnesium limitation may be beneficial to stimulate yeast oil production during the fermentation stage. Hence, the amount of magnesium in the growth medium may be selected to achieve a timely switch to oil generation, and thereby may facilitate achieving a particularly high oil composition yield.

A singular nitrogen limitation or magnesium limitation may result in improved yeast oil production. A combined nitrogen and magnesium limitation may result in even further improved yeast oil production. Hence, the preparation stage may comprise providing a growth medium such that a combined nitrogen and magnesium limitation will occur during the fermentation stage to facilitate obtaining a high oil composition yield.

Hence, in embodiments, the growth medium may comprise 0.00001-0.05 wt % of the magnesium source, i.e., the amount of the magnesium source in the growth medium may be in the range of 0.00001-0.05 wt %. In further embodiments, the amount of the magnesium source in the growth medium may be in the range of 0.00002-0.05 wt %, such as 0.00002-0.02 wt %. In embodiments, the amount of the magnesium source in the growth medium may be in the range of 0.00005-0.02 wt %, especially 0.00005-0.01 wt %. Moreover, the amount of magnesium source in the growth medium may be in the range of 0.0001-0.01 wt %, such as 0.0001-0.005 wt %. Further, the amount of magnesium source in the growth medium may be in the range of 0.0005-0.005 wt %, especially 0.0005-0.001 wt %.

In particular, the method, especially the preparation stage, may comprise providing the growth medium with an amount of the magnesium source suitable to arrive at a target cell density (also see below) during the fermentation stage, especially to (essentially) stop growth (due to a magnesium limitation) at a target cell density, such as of at least 5E7 cells/ml, during the fermentation stage.

The growth medium may, in embodiment, be a liquid medium. Hence, a substantial proportion of the growth medium may comprise water. For instance, in embodiments, the growth medium may comprise 0.25-25 wt % dry matter on basis of the total weight of the growth medium, i.e., the amount of dry matter in the growth medium may be in the range of 0.25-25 wt % based on the total weight of the growth medium. In further embodiments, the amount of dry matter in the growth medium may be in the range of 0.5-25 wt %, such as 0.5-15 wt %. In embodiments, the amount of dry matter in the growth medium may be in the range of 1-15 wt %, especially 1-10 wt %. In particular, an amount of dry matter in the range of up to 10 wt % may be particularly beneficial as higher amount may hamper stirring, which may in turn lead to a less efficient fermentation. Moreover, the amount of dry matter in the growth medium may be in the range of 2-10 wt %, such as 2-8 wt %. Further, the amount of dry matter in the growth medium may be in the range of 3-8 wt %, such as 3-6 wt %.

The term “dry matter” may herein especially refer to the dry weight relative to total weight. Hence, the phrase “the growth medium comprises at least 10 wt % dry weight” may indicate that the growth medium comprises a liquid content (or “moisture content”) of at most 90 wt %.

Besides the dry matter, the growth medium may comprise liquid content. In embodiments, the growth medium may comprise water. Generally, the liquid content of the growth medium may especially be mostly water, such as at least 95% of the liquid content of the growth medium may comprise water, especially at least 98%.

In embodiments, the liquid content of the growth medium may be provided by the biomass. Especially, the water content of the growth medium may be provided by the biomass. In some embodiments, relative to a total volume of the growth medium, at least 50 vol. % of the water content of the growth medium may be provided by the biomass, such as at least 75 vol. %, especially at least 90 vol. %. In further embodiments, at least 95 vol. % of the water content of the growth medium may be provided by the biomass, such as at least 98 vol. %, especially at least 99 vol. %. Moreover, (essentially) all of the water content of the growth medium may be provided by the biomass. Therefore, in embodiments, at maximum 1 vol. % water in the growth medium is added water, such as at maximum 2 vol. %, especially at maximum 5 vol. %. Hence, water costs and energy costs (from separating the water phase from the yeast oil in the oil procurement stage) may be reduced.

Furthermore, such embodiments may require relatively little to no additional water during the fermentation stage. In such embodiments, relative to a total volume of the cell suspension, at most 10 vol. % water may be added to the cell suspension during the fermentation stage, such as at most 7.5 vol. % water, especially at most 5 vol. %. Further, at most 3 vol. % water may be added to the cell suspension during the fermentation stage, such as at most 2 vol. % water, especially at most 1 vol. % water. Moreover, (essentially) no water may be added to the cell suspension during the fermentation stage. Thereby, water costs and energy costs (from separating the water phase from the yeast oil in the oil procurement stage) may be reduced.

Embodiments wherein the majority (or (essentially) all) of the water content is provided by the biomass and/or relatively little (or (essentially) no) additional water is added during the fermentation stage may result in a more economically feasible method by reducing water needs (and associated costs). Additionally, such embodiments may be more economically feasible by reducing the energy costs associated with separating the yeast oil from the water phase to provide the oil composition during the oil procurement stage (described further below). This may especially be advantageous for simplifying the invention and providing a more accessible and economically feasible method when combined with other manners of simplifying the invention as described herein, e.g. use of non-pasteurized (especially non-heated) biomass, not monitoring or controlling oxygen saturation and/or pH levels, and not adding additional nutrients during the fermentation stage.

The growth medium may have a pH that is suitable for yeast growth and yeast oil production during the fermentation stage. At a pH that is either too low (too acidic) or too high (too basic) the yeast cell may not achieve sufficient yeast growth and/or yeast oil production. Hence, the preparation stage may comprise setting the pH of the growth medium. In embodiments, the pH may change due to yeast growth and yeast oil production during the fermentation stage. Yet the pH may in embodiments not be controlled or adjusted during the fermentation stage.

Hence, the pH of the growth medium may be selected from the range of 2.0-9.0. In further embodiments, the pH of the growth medium may be selected from the range of 3.0-9.0, such as from the range of 3.0-8.0. In embodiments, the pH of the growth medium may be selected from the range of 4.0-8.0, especially from the range of 4.0-7.5. Moreover the pH of the growth medium may be selected from the range of 4.5-7.5, such as from the range of 4.5-7.0. Further, the pH of the growth medium may be selected from the range of 5.0-7.0, especially from the range of 5.0-6.5.

In the method of the invention, the yeast cell may produce relatively low amounts of acid, such as citric acid, during the fermentation stage. Especially, a relatively low oxygen saturation of the cell suspension (see below) may result in the production of relatively low amounts of (citric) acid. Hence, while prior art fermentations may typically involve pH control, pH control during the fermentation stage may be avoided with the method of the invention. In particular, a pH of the growth medium in the range of 4.0-8.0 may provide suitable conditions for yeast growth and yeast oil production without necessitating the need for further pH adjustment during later stages of the process, thereby simplifying the overall process. In particular, a suitable selection of the starting conditions of the growth medium and the aeration conditions may result in a suitable pH for growth and/or oil production by the yeast cell throughout the fermentation stage.

In embodiments, the method may comprise the fermentation stage. The fermentation stage may especially comprise growing yeast cells in the growth medium, as well as providing oils.

In embodiments, the fermentation stage may begin by inoculating the growth medium with a yeast cell. The term “yeast cell” may herein also refer to a plurality of yeast cells. The yeast cell may be in a liquid suspension form. In other embodiments, the yeast cell may be in a dried powder form. The yeast cell may in certain embodiments be obtained as a yeast cell isolate (described further below). In other embodiments, the yeast cell may be obtained as a yeast cell isolate commercially. In certain embodiments, the yeast cell may be obtained from a sample of a previous fermentation cycle according to the present invention.

Through inoculation of the growth medium, an (oleaginous) cell suspension may be provided. Hence, the term “cell suspension” may herein especially refer to the growth medium with the added yeast cell, including the subsequent cell growth during the fermentation stage. The yeast cell may be dispersed through the cell suspension. During the fermentation stage, the yeast cell may metabolize the nutrient sources, such as the carbon source, the nitrogen source, and the magnesium source. This metabolism may facilitate yeast growth, wherein the yeast cell obtains the nutrients necessary for cellular division and enters cellular multiplication. Hence, the number of yeast cells may increase during the fermentation stage and the cell density in the cell suspension may increase. Depending on the availability of nutrient sources, the increase in yeast cells may be exponential or linear.

The metabolism of nutrient sources by the yeast cell may further facilitate yeast oil production, wherein the yeast cell obtains the nutrients necessary for yeast oil production. The yeast cell may produce yeast oil under growth conditions, but may especially increase yeast oil production under conditions of (specific) nutrient limitations, such as nitrogen limitation and/or magnesium limitation. Yeast oil may be especially saved within the yeast cell, but some amount of yeast oil may also enter the cell suspension. Hence, the amount of yeast oil in the cell suspension may increase during the fermentation stage, especially in the yeast cell.

The temperature of the cell suspension during the fermentation stage may affect both the growth rate of the yeast cell and the oil production by the yeast cell. In particular, the profile, such as the fatty acid profile, of the yeast oil may vary with temperature. It will be clear to the person skilled in the art that the relation between temperature and growth rate as well as the relation between temperature and oil production will be dependent on the specific yeast that is used. For instance, Yarrowia lipolytica may grow well at temperatures in the range of 20-30° C. In contrast, temperatures above about 40° C. may be damaging or even deadly for the Yarrowia lipolytica, and metabolism of Yarrowia lipolytica may become rather slow with temperatures below 10° C.

Especially, the temperature of the cell suspension during the fermentation stage may affect the yeast oil production. The yeast cell may produce yeast oil with different fatty acid compositions at different temperatures of the cell suspension (further described below). Hence, by controlling the temperature during the fermentation stage, a yeast oil may be produced with the desired fatty acid composition. Especially, the temperature may be controlled during a nutrient limitation substage of the fermentation stage where the majority of yeast oil production occurs (as further described below). Hence, by controlling the temperature at the time when the majority of yeast oil is produced, the oil composition may have the desired fatty acid composition. The temperature of the cell suspension may be controlled in the range of 10-40° C. In certain embodiments, the temperature of the cell suspension may be controlled in the range of 10-24° C., such as in the range of 15-24° C. In further embodiments, the temperature of the cell suspension may be controlled in the range of 25-40° C., such as in the range of 25-35° C.

In embodiments, the nutrient abundance substage (see below) may comprise controlling a temperature of the cell suspension in the range of 20-30° C. In such embodiments, the nutrient limitation substage (see below) may especially comprise controlling a temperature of the cell suspension in the range of 10-20° C., or especially in the range of 30-40° C. In such embodiments, the yeast cell, especially the Y. lipolytica cell, may be grown at a temperature at which the yeast cell grows efficiently, but the yeast oil production may take place at a different temperature to steer the yeast oil towards a desired composition.

The yeast cell may especially be selected in view of its efficiency in converting the biomass, especially the first carbon source, into yeast biomass and into yeast oil. Hence, the yeast cell may be selected for the range of nutrient sources it can use, for having an (energy) efficient metabolism, and for generating relatively little by-products.

Hence, in embodiments, the yeast cell may be selected from the genera Yarrowia and Cutaneotrichosporon, especially from the genus Yarrowia, or especially from the genus Cutaneotrichosporon.

In further embodiments, the yeast cell may be selected from the species Yarrowia lipolytica, i.e., the yeast cell may comprise a Yarrowia lipolytica cell. Yarrowia lipolytica may be capable of using a broad range of carbon sources. Further, Yarrowia lipolytica may be used for the production of yeast oil with defined characteristics and has been classified as food grade (i.e. safe and suitable for consumption). Hence, Yarrowia lipolytica is particularly appropriate for the fermentation of biomass to provide an oil composition. Especially, strains of Yarrowia lipolytica may be selected that have desired yeast oil producing abilities.

In further embodiments, the yeast cell may be selected from the species Cutaneotrichosporon oleaginous. Cutaneotrichosporon oleaginous may be capable of growing and producing yeast oil at (relatively) low temperatures. Further, Cutaneotrichosporon oleaginous may be used for the production of yeast oil with defined characteristics and may provide particularly high yeast oil yields and/or yeast oil titers compared to other species of yeast. Hence, Cutaneotrichosporon oleaginous is also appropriate for the fermentation of biomass to provide an oil composition. Especially, strains of Cutaneotrichosporon oleaginous may be selected that have desired yeast oil producing abilities.

During the fermentation stage, the cell density and the yeast oil in the cell suspension will increase due to yeast growth. At a certain point, the yeast cell will have exhausted one or more nutrients required for further yeast growth and yeast oil production. At such point, the yeast oil may not further increase in the cell suspension and the oil may be harvested. Hence, in embodiments, the fermentation stage may be continued until the cell suspension reaches a (target) cell density selected from the range of 1E7-1E11 cells/ml, such as from the range of 1E8-1E10 cells/ml.

In further embodiments, the fermentation stage may be continued until the cell suspension reaches a (target) cell density of at least 5E6 cells/ml, such as at least 1E7 cells/ml, especially at least 5E7 cells/ml, such as at least 1E8 cells/ml.

In further embodiments, the fermentation stage may be continued until the yeast cell in the cell suspension reaches 0.4-20 wt %, i.e., 0.4-20 wt % of the cell suspension comprises the yeast cell (on dry weight basis). In further embodiments, the fermentation stage may be continued until the yeast cell in the cell suspension reaches 0.7-20 wt %, such as 0.7-15 wt %. In embodiments, the fermentation stage may be continued until the yeast cell in the cell suspension reaches 1-15 wt %, especially 1-10 wt %. Moreover the fermentation stage may be continued until the yeast cell in the cell suspension reaches 2-10 wt %, such as 2-5 wt %.

In some embodiments, the fermentation stage may be continued until the cell suspension has an optical density at 600 nm (OD600) of at least 20.

The produced yeast oil during the fermentation stage may largely be comprised by the yeast cell(s) (intracellularly). Hence, in embodiments, the method may comprise releasing the yeast oil from the yeast cell(s).

In embodiments, the method may comprise the oil procurement stage. The oil procurement stage may comprise separating the yeast oil from (other components of) the cell suspension to provide the oil composition. The oil procurement stage may comprise mechanically lysing the cell suspension to provide a lysed cell suspension. As the yeast oil may be primarily stored in the yeast cell, the yeast oil may be released into the lysed cell suspension such that the yeast oil may be separated from the other components of the (lysed) cell suspension to provide the oil composition. For this purpose, in embodiments, the yeast cell may be mechanically lysed to release the yeast oil into the lysed cell suspension. In certain embodiments, physically disrupting the yeast cell may be sufficient to release the yeast oil into the lysed cell suspension.

In embodiments, the oil procurement stage may comprise a mechanical lysing substage. The mechanical lysing substage may especially comprise one or more of screw-pressing, bead beating, French pressing, and homogenizing, especially one or more of screw-pressing bead beating, and French pressing.

Screw-pressing may comprise a mechanical method for lysing the yeast cell as well as separating yeast oil from other components of the (lysed) cell suspension. Using a (revolving) screw-press device, the cell suspension may be exposed to increased pressure and temperature, leading to lysis of the yeast cell and decreasing the viscosity of the oil phase. As the oil phase may then have a lower relative viscosity and density compared to the water phase, it may pass through holes in the screw-press device while the water phase and remaining solids remain in the screw-press device. Thereby, the oil phase may be separated from the water phase and remaining solids (e.g., from the lysed cell suspension). Screw pressing may be particularly convenient as it may simultaneously both lyse the yeast cells and separate the yeast oil from water.

Bead beating may comprise the addition of inorganic solid beads to the cell suspension. Through agitation of the cell suspension comprising the beads, the mechanical contact with the yeast cell may result in cell wall rupture of the yeast cell and hence lysis.

French pressing may comprise the use of a piston to produce a highly pressurized cell suspension. The highly pressurized cell suspension may then squeeze past a valve, during which the highly pressurized cell suspension may be exposed to shear stress and decompression. Hence, the yeast cell may be disrupted and lysed.

Homogenization may comprise various techniques that homogenize a biomass, including e.g. blender homogenization, sonication, rotor-stator mechanical homogenization, high pressure homogenization, etc. Especially, in embodiments, homogenization may comprise high pressure homogenization. The cell suspension may thus be exposed to pressures high enough to result in yeast cell disruption and lysis.

Herein, the oil procurement stage may further comprise a drying substage. During the drying substage, the cell suspension may be dried to comprise 70-97 wt % dry weight content (or “dry matter”), i.e., the cell suspension may be dried such that 70-97 wt % of the total weight of the cell suspension is dry weight. Drying may comprise exposing the cell suspension to drying conditions leading to evaporation of water. Such drying conditions may comprise high temperatures and low humidity air. High temperatures may herein comprise temperatures over 40° C., like over 50° C., especially over 60° C., up to over 80° C. Low humidity air may comprise air with a relative humidity below 40%, like below 30%, especially below 20%, up to below 10%. In embodiments, such a drying substage may precede the mechanical lysing. Especially, such a drying substage may precede screw-pressing.

In embodiments, the cell suspension may be dried to comprise 50-100 wt % dry weight content on basis of the total weight of the cell suspension, i.e., the amount of dry weight content in the cell suspension may be in the range of 50-100 wt %, such as in the range of 50-99 wt %. In further embodiments, the cell suspension may be dried to comprise dry weight content in the range of 60-99 wt %, such as 60-98 wt %. In embodiments, the cell suspension may be dried to comprise dry weight content in the range of 70-98 wt %, especially 70-97 wt %. Moreover, the cell suspension may be dried to comprise dry weight content in the range of 75-97 wt %, such as 75-95 wt %. Further, the cell suspension may be dried to comprise dry weight content in the range of 80-95 wt %, such as 80-90 wt %.

In particular, higher amounts of dry weight content may be particularly beneficial for screw-pressing, as this may lead to higher oil yields. However, drying the cell suspension to high amounts of dry weight content may require time and energy, with diminishing results at higher amounts of dry weight content. Hence, in embodiments the amount of dry weight content in the cell suspension may represent a balance between the advantage of higher oil yields and the disadvantage of time and energy expenditure.

Hence, in embodiments, the drying stage may comprise drying the cell suspension to a liquid content (or “moisture content”) of at most 30 wt %, such as of at most 25 wt %, especially at most 20 wt %. In further embodiments, the drying stage may comprise drying the cell suspension to a liquid content of at most 15 wt %, such as of at most 10 wt %, especially at most 5 wt %. In further embodiments, the drying stage may comprise drying the cell suspension to a liquid content of at least 3 wt %, such as of at least 5 wt %, especially at least 10 wt %.

In embodiments, the oil procurement stage may further comprise a separation substage comprising separating the oil composition from (remaining components of) the lysed cell suspension. In particular, the lysed cell suspension may comprise solid residue, such as biomass residue, being all the solid components of the biomass in the growth culture that were not metabolized by the yeast cell during the fermentation stage, and such as cellular debris, being the solid components of the yeast cell, such as e.g. proteins. The lysed cell suspension may further comprise a water phase. The water phase may comprise liquid from the growth culture, water-soluble compounds from the growth culture, and water-soluble compounds from the yeast cell. Hence, the separation substage may comprise separating the yeast oil from the solid residue and the water phase to provide the oil composition. In particular, the oil composition may comprise the yeast oil, and optionally oil-soluble compounds from the growth medium, and oil-soluble compounds from the yeast cell.

Such a separation substage may follow the mechanical lysing comprising one or more of screw-pressing, bead beating, French pressing, and homogenizing. The separation substage may especially follow the mechanical lysing in embodiments where the mechanical lysing comprises one or more of bead beating, French pressing, and homogenizing. In embodiments where the mechanical lysing comprises screw-pressing, the yeast oil may be separated from the other components of the lysed cell suspension such that a separation substage may not be required to provide the oil composition. In other embodiments where the mechanical lysing comprises screw-pressing, the separation substage may be an additional step following the yeast oil separation by the screw-pressing to provide a higher quality oil composition with fewer residual components from the cell suspension.

Especially, in embodiments of the present invention, the oil procurement stage may be devoid of chemical lysing. For instance, no lysing agents may be added to the (lysed) cell suspension or the supernatant.

Hence, in embodiments wherein the mechanical lysing comprises screw-pressing, the cell suspension may especially (first) be dried in a drying substage. The yeast oil may then be separated from cellular debris, biomass residue, and water in the cell suspension during the screw-pressing.

In other embodiments wherein the mechanical lysing comprises one or more of bead beating, French pressing, and homogenizing, the oil procurement stage may especially further comprise a separation substage comprising centrifuging the lysed cell suspension to provide a supernatant and a pellet. The separation substage may comprise separating the yeast oil from water in the supernatant to provide the yeast composition.

Hence, the method of the invention may provide a process for turning (plant) biomass into an oil composition by fermenting the biomass using a yeast cell, and subjecting a resulting cell suspension to mechanical lysis and oil separation. Biomass, especially biomass comprising (insoluble) fibers, such as biomass comprising (unprocessed) plant material, may provide an extra benefit due to the presence of fibers as extra solids. These may provide extra mechanical resistance in the physical extraction of the oil from the fermented biomass and hence result in higher oil composition yields. However, high amounts of such fibers may complicate stirring and/or aeration during the fermentation stage. Hence, in embodiments, the biomass, especially the dry matter, may comprise (insoluble) fibers. In further embodiments, the growth medium may comprise 0-5 wt % (insoluble) fibers, such as 0.01-5 wt %, especially 0.1-5 wt %.

The (initial) biomass may be obtained from various sources. For instance, the production of primary food products and primary agricultural products may result in various streams of biomass which may be suitable for the present invention. In embodiments, the biomass may comprise one or more of a side stream, a waste stream, and a residual stream. Such streams of biomass may, for instance, be obtained from one or more of the food industry and agricultural industry. Biomass from such streams may often be discarded or disposed. The present invention may provide a suitable alternative for the use of material that would otherwise not be used rather than, for instance, relying on food-grade biomass. In certain embodiments, the present invention may provide a more economically attractive option than other alternative methods for the use of such biomass.

Primary products may be the dominant value products of a production process, e.g. vegetables and fruits may be grown and harvested with the express purpose of using said vegetables and fruits as a food product. Such vegetables and fruits may especially include e.g. broccoli, avocado, tomato, cabbage, bell pepper, carrot, kale, lettuce, cucumber, spinach, chard, mangold beet, napa cabbage, endive, potato, beetroot, artichoke, citrus fruits, apples, grapes, etc. Under certain circumstances, such as e.g. market forces, quality demands, or decomposition, the primary products may lose significant economic value. The primary product may thus become available as the residual stream of the production process and may, for instance, be suitable as biomass for the method of the invention. Hence, in embodiments, the biomass may comprise a residual stream. Especially, in embodiments, the biomass may comprise a vegetable or fruit, especially one or more of broccoli, avocado, tomato, cabbage, bell pepper, carrot, kale, lettuce, cucumber, spinach, chard, mangold beet, napa cabbage, endive, potato, beetroot, artichoke, citrus fruits, apples, and grapes.

Production processes from the food industry and agricultural industry may further result in a side stream of secondary products. Secondary products may not comprise the dominant value products of a production process but may comprise by-products of the process that retain economic value. The production process may in certain processes include elements and steps to optimize or refine secondary products depending on their economic value. Such secondary products may especially include e.g. brewer's spent-grain from beer brewing and production of other alcoholic beverages, press mud from the sugar refinery process of sugar beet and sugar cane, solid waste from the plant and herbal extraction process, crude glycerol from the biofuel and vegetable oil production process. Such secondary product may become available as the side stream of the production process and may be suitable as a source material for the biomass. Hence, in embodiments, the biomass may comprise a side stream.

Production processes from the food industry and agricultural industry may additionally result in a waste stream of waste products. Waste products may be by-products of the process that contain little to no economic value and are commonly discarded or disposed of. Such waste products may especially include e.g. peelings, cuttings, and scrapings from vegetables and fruits such as those described above, and non-edible parts of the plant that may be taken along during the harvesting process. Such waste product may thus become available as the waste stream of the production process and may hence be suitable as a source material for biomass. Hence, in embodiments, the biomass may comprise a waste stream.

Biomass from the streams mentioned above may upon generation during their production process(es) typically have economic value, especially with regards to the side stream and residual streams. However, in certain circumstances the biomass may not be sold or transported on time and a natural decomposition process may start. As a result of the natural decomposition process, the biomass may rapidly decline in economic value, for instance due to unsuitability as a food source, and may instead be considered a part of the waste stream. The present invention may be applicable for biomass that is (partly) decomposed and may hence provide a viable alternative for (waste) material that would otherwise be disposed. Hence in embodiments, the biomass may comprise (at least partially) decomposed biomass.

The natural decomposition process may comprise metabolism of nutrients in the biomass by bacteria, protozoa, fungi, and animals, especially of a carbon source in the biomass. The carbon source may be metabolized into a decomposition compound, especially by bacteria, protozoa, and fungi. Such a decomposition compound may, for instance comprise at least one or more from the group comprising ethanol, acetic acid, and lactic acid. The term “decomposition compound” may also refer to a plurality of (different) decomposition compounds.

The use of a (partially) decomposed biomass in the method of the invention may be particularly beneficial in view of a circular economy and sustainability. Especially, the yeast cell may be able to ferment the decomposition compound and metabolize it as a nutrient source, while the decomposition compound may typically not be bioavailable as a nutrient source to other organisms. Hence, in embodiments, the biomass may comprise decomposed biomass. For instance, the decomposed biomass may comprise 0.01-10 wt % of a decomposition compound. In further embodiments, the decomposed biomass may comprise 0.02-10 wt % of a decomposition compound, such as 0.02-5 wt %. In embodiments, the decomposed biomass may comprise 0.05-5 wt % of a decomposition compound, especially 0.05-2 wt %. Moreover the decomposed biomass may comprise 0.1-2 wt % of a decomposition compound, such as 0.1-1 wt %.

In further embodiments, at least 0.01 wt % of the decomposed biomass comprises the decomposed biomass, especially at least 0.02 wt %, such as at least 0.05 wt %. In further embodiments, at least 0.1 wt % of the decomposed biomass comprises the decomposition compound, especially at least 0.2 wt %.

In the decomposed biomass, 0.02-20 wt % of (initial) carbon sources may have been converted to the decomposition compound. In further embodiments, 0.05-20 wt % of the carbon sources may have been converted to the decomposition compound, such as 0.05-10 wt %.

The decomposition compound may especially serve as a carbon source for the yeast cell, i.e., the carbon source may comprise the decomposition compound.

In further embodiments, at least 3 wt % of the carbon source may comprise the decomposition compound, such as at least 5 wt %, especially at least 10 wt %. In further embodiments, at least 15 wt % of the carbon source may comprise the decomposition compound, such as at least 20 wt %, especially at least 30 wt %, including 100%. In further embodiments, at most 90 wt % of the carbon source may comprise the decomposition compound, such as at most 80 wt %, especially at most 50 wt %.

Biomass may typically be pasteurized prior to a fermentation process to prevent (further) decomposition, and to prevent competing microbial processes. However, pasteurization may be an energy-intensive process and may require specialized equipment.

However, the method of the invention may be compatible with non-pasteurized biomass as a high inoculum of the yeast cell may be able to outgrow and outcompete wild-type (micro-)organisms present in the non-pasteurized biomass. Further, in such embodiments where the growth medium has a relatively low pH, such as lower than 5.0, especially lower than 4.0, moreover lower than 3.0, the yeast cell may be able to outgrow and outcompete wild-type (micro-)organisms in the non-pasteurized biomass with a diminished ability to grow and metabolize at such pH levels. This may especially be advantageous for simplifying the invention and providing a more accessible and economically feasible method when combined with other manners of simplifying the invention as described herein, e.g. not monitoring or controlling oxygen saturation and/or PH levels, and not adding additional nutrients and/or water during the fermentation stage.

In embodiments, the fermentation stage may comprise inoculating the growth medium with a yeast cell inoculum comprising the yeast cell. In particular, the yeast cell inoculum may have an inoculum cell density selected from the range of 1E8-1E10 cells/ml, such as of (about) 1E9. In further embodiments, the fermentation stage may comprise adding an amount of the yeast cell inoculum to the growth medium selected from the range of 0.1-10 vol % of the growth medium, such as from the range of 0.2-5 vol % of the growth medium, especially from the range of 0.5-1 vol % of the growth medium.

Hence, in embodiments, the biomass may comprise a non-pasteurized biomass. In particular, in embodiments, the biomass may especially comprise a non-heated biomass, such as wherein the non-heated biomass has not been exposed to a temperature of at least 45° C., such as especially at least 63° C., for at least 5 minutes, such as especially at least 10 minutes. Use of a non-heated biomass may yet further simply the execution of the invention and be especially advantageous when combined with other manners of simplifying the invention as described herein.

The growth medium may in embodiments comprise at least one or more supplementary nutrient sources to facilitate yeast growth and yeast oil production. The supplementary nutrient source may be at least partly provided by the biomass. Especially, the supplementary nutrient source may be entirely provided by the biomass. In other embodiments, the supplementary nutrient source may be supplemented to the growth medium. Such supplementary nutrient sources may be present in relatively small quantities while facilitating suitable conditions for yeast growth and yeast oil production.

In embodiments, the supplementary nutrient source may comprise a salt. Especially, the salt may comprise at least one or more ions selected from the groups of phosphates, sulphates, sodium, and potassium. The growth medium may comprise 0.01-4 wt % of the salt. The growth medium may further comprise 0.02-4 wt % of the salt, such as 0.02-2 wt %. In embodiments, the growth medium may comprise 0.05-2 wt % of the salt, especially 0.05-1 wt %. Moreover, the growth medium may comprise 0.1-1 wt % of the salt, such as 0.1-0.5 wt %.

In further embodiments, the supplementary nutrient source may comprise a trace element. Especially, the trace element may comprise one or more elements selected from the groups of iron [Fe], calcium [Ca], manganese [Mn], zinc [Zn], and cobalt [Co]. The growth medium may especially comprise 0.0001-0.04 wt % of the trace element. The growth medium may further comprise 0.0002-0.04 wt % of the trace element, such as 0.0002-0.02 wt %. In embodiments, the growth medium may comprise 0.0005-0.02 wt % of the trace element, especially 0.0005-0.01 wt %. Moreover, the growth medium may comprise 0.001-0.01 wt. ‰ of the trace element, such as 0.001-0.005 wt %.

In embodiments, the fermentation stage may have a duration selected from the range of 1-12 days, especially from the range of 1.5-12 days, such as from the range of 1.5-10 days. In further embodiments, the fermentation stage may have a duration selected from the range of 2-10 days, especially from the range of 2-8 days, such as from the range of 2.5-8 days, especially from the range of 2.5-6 days. In particular, a short fermentation stage may be preferred as the risk of contamination by other (micro-) organisms may be decreased and may provide more economical use of the equipment available for fermentation such as e.g. the fermentation vessel.

Generally, the method of the invention may comprise providing a suitable growth medium for the entire fermentation stage in one go. However, in embodiments, the fermentation stage may comprise periodically adding a second carbon source to the cell suspension. The second carbon source may be selected from the group comprising glucose, maltose, ethanol, lactose, sucrose, galactose, fructose, lactic acid, acetic acid, propionic acid, butyric acid, and citric acid. The second carbon source may be added during the fermentation stage to provide the yeast cell with additional nutrients for yeast growth and yeast oil production. In embodiments, the fermentation stage may comprise providing the second carbon source by providing a second biomass. The second biomass may comprise material that came available from one of the streams described above at a later time than the (first) biomass. Hence, the second biomass may be added to the cell suspension during the fermentation stage and may contribute to the fermentation process, which may be a cost-effective and easy method of achieving a higher oil composition yield while effectively utilizing additional biomass material (optionally procured at a later time). In particular, growth may initially be continued until it stops because of nitrogen and/or magnesium limitation and then extra carbohydrate/sugar may be added for (extra) oil production.

The second carbon source may be added to the cell suspension at the range of 0.05-20 wt % (based on dry weight of the cell suspension), i.e., after addition of the second carbon source to the cell suspension, 0.05-20 wt % of the cell suspension may comprise the second carbon source on dry weight basis. The second carbon source may further be added to the cell suspension at the range of 0.1-20 wt %, such as 0.1-10 wt %. In embodiments, the second carbon source may be added to the cell suspension at the range of 0.2-10 wt %, especially 0.2-5 wt %. Moreover, the second carbon source may be added to the cell suspension at the range of 0.5-5 wt %, such as 0.5-2 wt %.

The second carbon source may be added to the cell suspension at an interval selected from the range of 6-48 hours. The second carbon source may further be added to the cell suspension at an interval selected from the range of 12-48 hours, such as selected from the range of 12-36 hours. In embodiments, the second carbon source may be added to the cell suspension at an interval selected from the range of 18-36 hours, especially selected from the range of 18-30 hours.

The fermentation stage may further comprise controlling a temperature of the cell suspension. Especially, the temperature of the cell suspension may be controlled to provide an oil composition with desired characteristics, as the yeast cell may produce yeast oil with different characteristics depending on the temperature of the cell suspension during the fermentation stage. Hence, in embodiments, the fermentation stage may comprise controlling a temperature of the cell suspension in the range of 10-24° C., such as 15-24° C., especially 20-24° C. Thus, the fermentation stage may in such embodiments proceed under (relatively) low temperatures. Yet in other embodiments, the fermentation stage may comprise controlling a temperature of the cell suspension in the range of 25-40° C., such as 25-35° C., especially 25-30° C. Thus, the fermentation stage may in such embodiments proceed under (relatively) high temperatures.

This may facilitate providing an oil composition with desired oil characteristics. Oil characteristics that may be controlled through temperature control may especially comprise the carbon chain length and carbon chain saturation of the fatty acids comprised by the oil composition. These two fatty acid properties may affect the oil composition and the application of the oil composition in products. Hence, the present invention provides an effective method of controlling the fatty acid carbon chain characteristics to provide an oil composition with characteristics that make it suitable for processing into products.

For instance, table 1 summarizes oil compositions obtained with fermentations using Yarrowia lipolytica or Cutaneotrichosporon oleaginous, the fermentation conditions only differing in the imposed temperature:

| Species | Temperature | SFA (%) | MUFA (%) | DUFA (%) | C16 (%) | C18 (%) |
|---|---|---|---|---|---|---|
| Yarrowia | 30° C. | 31% | 47% | 22% | 34% | 66% |
| lipolytica | 15° C. | 15% | 70% | 16% | 30% | 66% |
| Cutaneotrichosporon | 36° C. | 37% | 50% | 12% | 33% | 66% |
| oleaginous | 15° C. | 28% | 49% | 22% | 22% | 77% |

Vegetable oils may commonly comprise a majority of fatty acids with carbon chain lengths of 16 (C16) or carbon chain lengths of 18 (C18) in varying proportions, with vegetable oils commonly having a majority C18 over a minority C16 component. With certain yeast strains, the fermentation stage controlled at high temperature range may provide an oil composition with increased proportions of C16 and decreased proportions of C18 compared to fermentation stage controlled at low temperature range. With yet other yeast strains, the relative proportion of C16 and C18 may remain (essentially) the same at high temperature range and low temperature range.

Vegetable oils may further commonly comprise a majority of fatty acids with saturated (SFA), monounsaturated (MUFA), or diunsaturated (DUFA) carbon chains in varying proportions, with vegetable oils commonly having a majority C1 over minority SFA and DUFA components. With certain yeast strains, the fermentation stage controlled at high temperature range may provide an oil composition with increased proportions of SFA and DUFA and decreased proportions of MUFA compared to fermentation stage controlled at low temperature range. With yet other yeast strains, the fermentation stage controlled at high temperature range may provide an oil composition with increased proportion of SFA and decreased proportion of DUFA compared to fermentation stage controlled at low temperature range.

In embodiments, the fermentation stage may comprise controlling the temperature of the cell suspension in a same temperature range throughout the duration of the fermentation stage, e.g. in the temperature range of 10-24° C., or in the temperature range of 25-40° C. In certain embodiments, the temperature range of the cell suspension may be controlled through at least a part of the fermentation stage. The temperature range of the cell suspension may be controlled at different temperatures during different parts of the fermentation stage. In specific embodiments, the temperature range of the cell suspension may first be controlled to a low temperature range, and later at a high temperature range, or vice versa.

In embodiments, the fermentation stage may be executed in a fermentation vessel. A fermentation vessel may be selected from the group comprising a fermentation tank, a fermentation vat, a fermentation barrel, or further options. In particular, the fermentation vessel may be open at one or more sides, i.e., the fermentation vessel may be open to ambient air. In other embodiments, the fermentation vessel may be closed at all sides to avoid exposure to the external environment. For instance, in embodiments, the fermentation vessel may comprise a simple fermentation vessel, such as a barrel or a vat. In specific embodiments, the fermentation vessel may comprise or be functionally coupled to a compressed air supply and/or a stirring apparatus.

Especially, the fermentation stage may not comprise monitoring of oxygen saturation (or: “air saturation”) or pH level of the cell suspension. In prior art, the fermentation vessel may comprise specialized equipment such as a dissolved oxygen meter or a (potentiometric) pH meter. In embodiments, the fermentation vessel may not comprise such specialized equipment, and may especially not be configured to monitor the air saturation or pH level of the cell suspension during the fermentation stage. Embodiments of the current invention provide a method to provide air saturation and PH levels for yeast cell growth and yeast oil production without active monitoring in a simple fermentation vessel. Hence, the current invention may be accessible and economically feasible to perform as it may not require the use of such specialized equipment.

Moreover, in embodiments, the fermentation stage may not comprise controlling oxygen saturation or pH level of the cell suspension. In prior art, the fermentation vessel may comprise specialized equipment such as a stirring apparatus (optionally linked to a dissolved oxygen meter) or a pH pump (optionally linked to a pH meter). In embodiments, the fermentation vessel may not comprise such specialized equipment, and may especially not be configured to control the oxygen saturation or pH level of the cell suspension during the fermentation stage. Embodiments of the current invention provide a method to provide air saturation and PH levels for yeast cell growth and yeast oil production without active control in a simple fermentation vessel. Hence, the current invention may be yet more accessible and economically feasible to perform as it may not require the use of such specialized equipment. Some embodiments may comprise not controlling oxygen saturation of the cell suspension. Other embodiments may comprise not controlling pH level of the cell suspension. Specific embodiments may comprise not controlling both oxygen saturation and PH level of the cell suspension. Therefore, the method may have a particularly low barrier of entry and be more accessible and economically feasible (especially when combined with other manners of simplifying the invention as described herein, e.g. using a non-heated biomass and not adding additional nutrients and/or water during the fermentation stage).

In embodiments, the fermentation stage may comprise controlling an aeration in the cell suspension. In embodiments, the aeration may be achieved in a fermentation vessel exposed to open air. In further embodiments, the method may comprise providing an air flow to the cell suspension and/or stirring the cell suspension. Hence, in embodiments, the fermentation vessel may comprise or be functionally coupled to an air supply and/or a stirring apparatus, especially an air supply, or especially a stirring apparatus. The present invention may be applicable without the use of specialized fermentation equipment, such as a specialized fermentation vat. This may make the present invention relatively simple in use and economically attractive compared to alternative methods.

Yet the oxygen saturation may in certain embodiments not be controlled or adjusted during the fermentation stage (as described above). Especially, the oxygen saturation may in some embodiments not be controlled or adjusted by controlling an aeration in the cell suspension during the fermentation stage. Hence, the present invention may be even further simplified in terms of specialized equipment and may further reduce energy costs by removing the need for aeration of the cell suspension by an air supply and/or a stirring apparatus. Therefore, the method may have a lower barrier of entry and be more accessible (especially when combined with other manners of simplifying the invention as described herein).

In other embodiments, the oxygen saturation may be controlled (by aeration of the cell suspension) during at least part of the fermentation stage, and may not be controlled (by aeration of the cell suspension) during at least another part of the fermentation stage. In particular, the oxygen saturation may be controlled during the nutrient abundance substage. Furthermore, the oxygen saturation may not be controlled during the nutrient limitation substage. Hence, the method may be optimized in terms of oxygen saturation control of the cell suspension by providing a balance between reducing energy costs (by aerating the cell suspension) and increasing yeast lipid production.

In embodiments, the fermentation stage may comprise controlling an aeration in the cell suspension to provide 5-70% oxygen saturation in the cell suspension, such as 5-55% oxygen saturation, especially 5-30% oxygen saturation in the cell suspension, more especially 8-30% oxygen saturation, such as in the range of 8-25% oxygen saturation. In further embodiments, the fermentation stage may comprise controlling an aeration in the cell suspension to provide 10-25% oxygen saturation, especially 10-20% oxygen saturation, such as 12-20% oxygen saturation, especially 12-15% oxygen saturation. Achieving oxygen saturation in this range may provide beneficial conditions for yeast growth and yeast oil production, as it may provide sufficient oxygen for yeast cell growth during the nutrient abundance substage, but may provide an additional stress condition during the nutrient limitation substage resulting in improved yeast oil production. Further, a relatively low level of oxygen saturation may result in relatively little (citric) acid generation by the yeast cell, which may facilitate maintaining a suitable pH for yeast cell growth and oil production. Generally, higher oxygen saturation, such as 20% oxygen saturation, may result in improved yeast cell growth. The oxygen saturation levels in the present invention may be lower than those typically used for yeast cell growth and yeast oil production in the prior art. Hence, the present invention may allow for efficient yeast oil production with simpler techniques and lower oxygen saturation levels. This may especially be advantageous for simplifying the invention and providing a more accessible and economically feasible method when combined with other manners of simplifying the invention as described herein, e.g. using a non-heated biomass, not controlling oxygen saturation and/or pH level, and not adding additional nutrients and/or water during the fermentation stage

The aeration may be achieved through providing compressed air. The aeration may in other embodiments be achieved through continuous stirring. Especially, the aeration may be achieved through providing compressed air while simultaneously stirring continuously. Combining these aeration techniques may be especially effective for achieving the desired oxygen saturation.

Hence, in embodiments, the aeration may comprise providing a supply of compressed air to the fermentation vessel. The supply of compressed air to the fermentation vessel may be in the range of 2-30 L/min. The supply of compressed air to the fermentation vessel may further be in the range of 3-30 L/min, such as 3-25 L/min. In embodiments, the supply of compressed air to the fermentation vessel may be in the range of 5-25 L/min, especially 5-20 L/min. Moreover, the supply of compressed air to the fermentation vessel may be in the range of 8-20 L/min, such as 8-15 L/min.

The aeration may additionally comprise stirring (continuously). In particular, in embodiments, the fermentation stage may comprise stirring the cell suspension, especially at a rate selected from the range of 100-800 rpm, such as from the range of 200-400 rpm.

In embodiments, the fermentation stage may comprise two substages. The first substage may be a nutrient abundance substage, which may be followed by a nutrient limitation substage. Hence, the fermentation stage may comprise a nutrient abundance substage and a nutrient limitation substage. During the nutrient abundance substage, the cell suspension may comprise sufficient nutrients for yeast growth, particularly with regards to nitrogen and magnesium. As the yeast cell metabolizes these nutrients in the cell suspension for yeast growth, the nutrients will deplete from (free liquid of) the cell suspension. The cell suspension may then enter the nutrient limitation substage. Especially, during the nutrient limitation substage yeast cell growth may be substantially reduced relative to the nutrient abundance substage, while yeast oil production may continue and may be higher than during the nutrient abundance substage.

The nutrient limitation substage may have a nutrient limitation duration of at least 2 days, such as at least 4 days, or at least 6 days. In further embodiments, the nutrient limitation substage may have a nutrient limitation duration of at most 8 days, such as at most 7 days, especially at most 5 days, such as at most 3 days. In embodiments, the cell suspension may especially reach the nutrient limitation substage when reaching a certain cell density, such as a cell density selected from the range of 5E5-1E9 cells/ml, or especially from the range of 1E7-5E8 cells/ml.

During the nutrient limitation substage, the amount of (bioavailable) nitrogen and/or magnesium in the cell suspension may be insufficient to support further growth of the yeast cell.

In particular, in embodiments, during the nutrient limitation stage, the amount of the (bioavailable) nitrogen source in the cell suspension may be in the range of at most 0.5 wt %, such as at most 0.05 wt %, especially at most 0.001 wt %, including 0 wt %. In further embodiments, during the nutrient limitation substage, the amount of (bioavailable) nitrogen source in the cell suspension may be in the range of 0.001-0.5 wt %. In further embodiments, the amount of nitrogen source in the cell suspension may be in the range of 0.002-0.5 wt %, such as 0.002-0.2 wt %. In embodiments, the amount of nitrogen source in the cell suspension may be in the range of 0.005-0.2 wt %, especially 0.005-0.1 wt %. Moreover, the amount of nitrogen source in the cell suspension may be in the range of 0.01-0.1 wt %, such as 0.01-0.05 wt %.

Similarly, in embodiments, during the nutrient limitation substage, the amount of the magnesium source in the cell suspension may be in the range of at most 0.001 wt %, such as at most 0.00001 wt %, especially at most 0.000001 wt %, including 0 wt %. In further embodiments, during the nutrient limitation substage, the amount of (bioavailable) nitrogen source in the cell suspension may be in the range of 0.000001-0.01 wt %. In further embodiments, the amount of the magnesium source in the cell suspension may be in the range of 0.000002-0.05 wt %, such as 0.000002-0.002 wt %. In embodiments, the amount of the magnesium source in the cell suspension may be in the range of 0.000005-0.002 wt %, especially 0.000005-0.001 wt %. Moreover, the amount of the magnesium source in the cell suspension may be in the range of 0.00001-0.01 wt %, such as 0.00001-0.0005 wt %. Further, the amount of the magnesium source in the cell suspension may be in the range of 0.00005-0.0005 wt %, especially 0.00005-0.0001 wt %.

In embodiments, the right conditions for reaching the desired nutrient limitation substage at a desired yeast cell density may be achieved by combining different biomass sources. In other embodiments, a single biomass source may provide the right conditions for reaching the nutrient limitation substage at the desired yeast cell density.

The method, especially the nutrient limitation substage, may comprise providing a second carbon source to facilitate oil production (see above). In such embodiments, the second carbon source may be added immediately preceding or during the nutrient limitation substage. Hence, a nutrient limitation substage with a limitation in nitrogen source and magnesium but sufficient carbon source for oil production may be achieved.

The second carbon source may be added to the cell suspension during the nutrient limitation substage at the range of 0.05-20 wt % (based on dry weight of the cell suspension), i.e., after addition of the second carbon source to the cell suspension, 0.05-20 wt % of the cell suspension may comprise the second carbon source on dry weight basis. The second carbon source may further be added to the cell suspension during the nutrient limitation substage at the range of 0.1-20 wt %, such as 0.1-10 wt %. In embodiments, the second carbon source may be added to the cell suspension during the nutrient limitation substage at the range of 0.2-10 wt %, especially 0.2-5 wt %. Moreover, the second carbon source may be added to the cell suspension during the nutrient limitation substage at the range of 0.5-5 wt %, such as 0.5-2 wt %.

In embodiments, the fermentation stage may be continued until the cell suspension comprises at least 10 wt % of a yeast oil (on dry weight basis). The fermentation stage may further be continued until the cell suspension comprises at least 15 wt % of a yeast oil, such as at least 20 wt %. In embodiments, the fermentation stage may be continued until the cell suspension comprises at least 25 wt % of a yeast oil, especially at least 30 wt %. In particular, extraction of the oil from a cell suspension comprising at least 30 wt % oil may be particularly beneficial and may facilitate direct extraction of the oil from a cell suspension by screw-pressing. Moreover, the fermentation stage may be continued until the cell suspension comprises at least 35 wt % of a yeast oil, such as 40 wt %. This may especially be determined using the spectrophotometric vanillin method, such as described in Byreddy A R et al., “A quick colorimetric method for total lipid quantification in microalgae.” J Microbiol Methods. 2016 June; 125:28-32, which is hereby herein incorporated by reference. Hence, the fermentation stage may be concluded based on a suitable yield of oil composition.

The method may further comprise an oil procurement stage comprising separating the oil from (other components of) the cell suspension. The oil procurement stage may comprise subjecting the cell suspension to mechanical lysing to provide a lysed cell suspension, especially wherein the mechanical lysing comprises one or more of screw-pressing, bead beating, French pressing, and homogenization. In particular, different mechanical lysing methods may beneficially be applied for different yeasts.

For instance, in embodiments wherein the yeast cell comprises a Yarrowia lipolytica yeast cell, the mechanical lysing may especially comprise one or more of screw-pressing, bead beating, and French pressing. These methods have been described above and result in a higher oil recovery when applied to a cell suspension comprising Yarrowia lipolytica compared to homogenizing. Hence, these mechanical lysing methods may be particularly appropriate for effectively achieving high oil composition yields in embodiments involving Yarrowia lipolytica.

In other embodiments, during the cell lysis stage, wherein the yeast cell comprises a Cutaneotrichosporon oleaginous yeast cell, the mechanical lysing may especially comprise screw-pressing or French pressing. These methods have been described above and may result in a higher oil recovery when applied to a cell suspension comprising Cutaneotrichosporon oleaginous compared to bead beating and homogenizing.

Further, the mechanical lysing may in embodiments comprise a single lysis step. In other embodiments, the mechanical lysing may comprise at least two or more lysis steps. The lysis steps may be selected from at least two or more of screw-pressing lysis, bead beating lysis, French press lysis, and homogenization lysis.

The method, especially the oil procurement stage, may, in embodiments, comprise a separation substage. The separation substage may comprise separating the yeast oil from the (lysed) cell suspension, i.e., separating the yeast oil from other components of the (lysed) cell suspension. For instance, in embodiments, the separation substage may comprise separating the yeast oil from solid material using one or more of centrifugation and filtration. Hence, in embodiments, the separation substage may comprise subjecting the (lysed) cell suspension to one or more of centrifugation and filtration, especially to centrifugation, or especially to filtration.

In embodiments, the separation substage may comprise subjecting the (lysed) cell suspension to filtration using membranes through which fluid materials, including the yeast oil. may pass while the solid materials may be filtered. In further embodiments, other methods to separate the solid materials from the fluid materials of the lysed cell suspension may be used.

In further embodiments, the separation substage may comprise centrifuging the lysed cell suspension to provide a supernatant and a pellet. The lysed cell suspension may be centrifuged at a centrifugal force from the range of 250-20,000×g. The lysed cell suspension may further be centrifuged at a centrifugal force from the range of 500-20,000×g, such as from the range of 500-15,000×g. In embodiments, the lysed cell suspension may be centrifuged at a centrifugal force from the range of 1,000-15,000×g, especially from the range of 1,000-10,000×g. Moreover, the lysed cell suspension may be centrifuged at a centrifugal force from the range of 1,500-10,000×g, such as from the range of 1,500-8,000×g. The lysed cell suspension may further be centrifuged at a centrifugal force from the range of 2,000-8,000×g, such as from the range of 2,000-6,000×g. The centrifugation time may have a duration of 5-60 minutes, such as 10-50 minutes, especially 15-40 minutes, such as 20-30 minutes.

The pellet may especially comprise solid materials from the lysed cell suspension. The pellet may especially comprise cellular debris, such as for example protein aggregates. The pellet may further comprise biomass residue (from the initial biomass), such as for example fibrous material. The supernatant may especially comprise a water phase and an oil phase. The oil phase may especially comprise the yeast oil, such as at least 80 vol %.

Hence, in embodiments, the separation substage may comprise subjecting the lysed cell suspension to centrifugation to separate a supernatant and a pellet, and subsequently separating an oil phase from a water phase of the supernatant to provide the oil composition using a screw-press (method).

In further processing, the oil composition may be further refined to remove the other materials in the oil phase. Such further refinement processes may include filtration, distillation, chemical purification, etc.

In a further aspect, the current invention may provide an oil composition obtainable by the method as described above. The oil composition may comprise fatty acids with defined carbon chain properties. Especially, 10-30% of the fatty acids in the oil composition may be palmitic acid, or C16/SFA fatty acids. Further, 0-20% of the fatty acids in the oil composition may be palmitoleic acid, or C16/MUFA fatty acids. Moreover, 5-29% of the fatty acids in the oil composition may be stearic acid, or C18/SFA fatty acids. Additionally, 36-53% of the fatty acids in the oil composition may be oleic acid, or C18/MUFA fatty acids. Especially, 5-22% of the fatty acids in the oil composition may be linoleic acid, or C18/DUFA fatty acids.

In a further aspect, the current invention may provide a product comprising the oil composition obtainable by the method described above. The product may, in embodiments, be selected from the group comprising a food product, a cosmetic product, a cleaning product, a fuel, and a medical product.

In a further aspect, the current invention may provide a process for the production of a product, especially wherein the product is selected from the group comprising a food product, a cosmetic product, a cleaning product, a fuel, and a medical product. The process may in embodiments comprise (i) providing an oil composition using the method as described above, and (ii) processing the oil composition into the product. Especially, the oil composition may be used in the type of products that currently comprise vegetable oil, such as palm oil. In such applications, the main functionalities of the oil composition in the product may be selected from the group comprising (i) sensorial function, (ii) carrier function (of flavors, colors, antioxidants, etc.), (iii) emulsifier function, and (iv) preserver function.

Further, the current invention may employ an isolation method for providing an isolated yeast cell. Such isolation method may comprise a strain enrichment stage. During this strain enrichment stage, a first biomass sample may be exposed to an enrichment medium. Such an enrichment medium may especially comprise 0.1-2.5 vol. % of hexadecane. Hence, an enriched biomass sample may be provided that is particularly appropriate for the enrichment of Yarrowia lipolytica yeast cell. This strain enrichment stage may be followed by a colony growth stage. The colony growth stage may comprise incubating the enriched biomass sample on a selective agar plate. Such a selective agar plate may especially comprise (i) 0.01-2 wt % of tyrosine, such as especially 0.02-1 wt % of tyrosine, such as moreover 0.04-0.5 wt % of tyrosine, and (ii) 0.001-0.2 wt % of manganese [Mn²⁺], such as especially 0.002-0.1 wt % of manganese [Mn²⁺], such as moreover 0.004-0.05 wt % of manganese [Mn²⁺]. The colony growth stage may last a duration of 12-72 hours and may then be followed by a colony picking stage. A selective agar plate comprising tyrosine and manganese may provide Yarrowia lipolytica yeast cell as red/brown colony. The colony picking stage may comprise picking a red/brown colony from the selective agar plate to provide the isolated yeast cell.

In embodiments, the method may comprise providing the yeast cell using the isolation method. Especially, the isolated yeast cell may be selected from the species Yarrowia lipolytica.

In further embodiments, the yeast cell may comprise the isolated yeast cell.

The embodiments described herein are not limited to a single aspect of the invention. For example, an embodiment describing the method may, for example, further relate to the oil composition provided by the method, and to a product made from the oil composition. Similarly, an embodiment of the isolation method describing the (isolated) yeast cell may further relate to the yeast cell used in the method.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1A-B schematically depict embodiments of the method for producing an oil composition from biomass.

FIG. 2 schematically depicts embodiments comprising a nutrition abundance substage and nutrition limitation substage.

FIG. 3A-B depict experimental results obtained with embodiments of the method for producing an oil composition from biomass. The schematic drawings are not necessarily on scale.

### DETAILED DESCRIPTION OF THE EMBODIMENTS

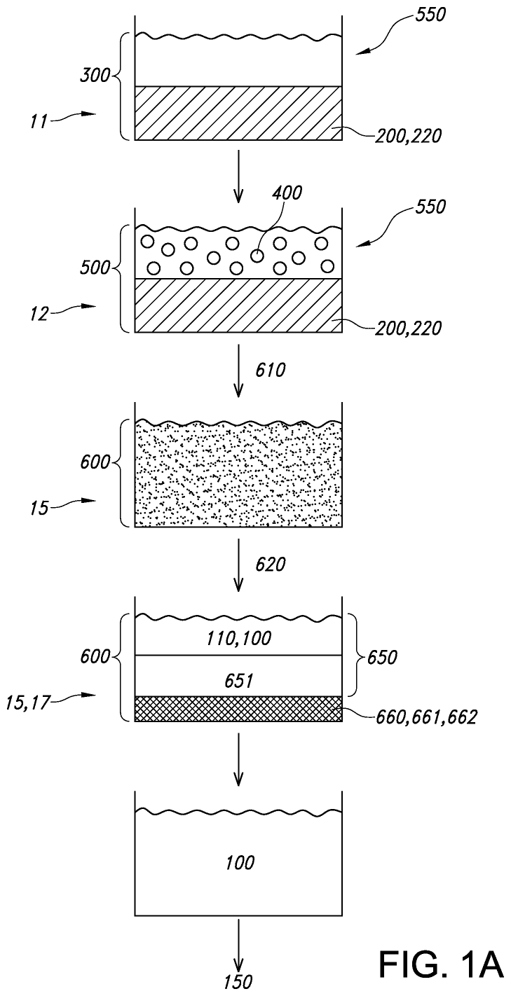

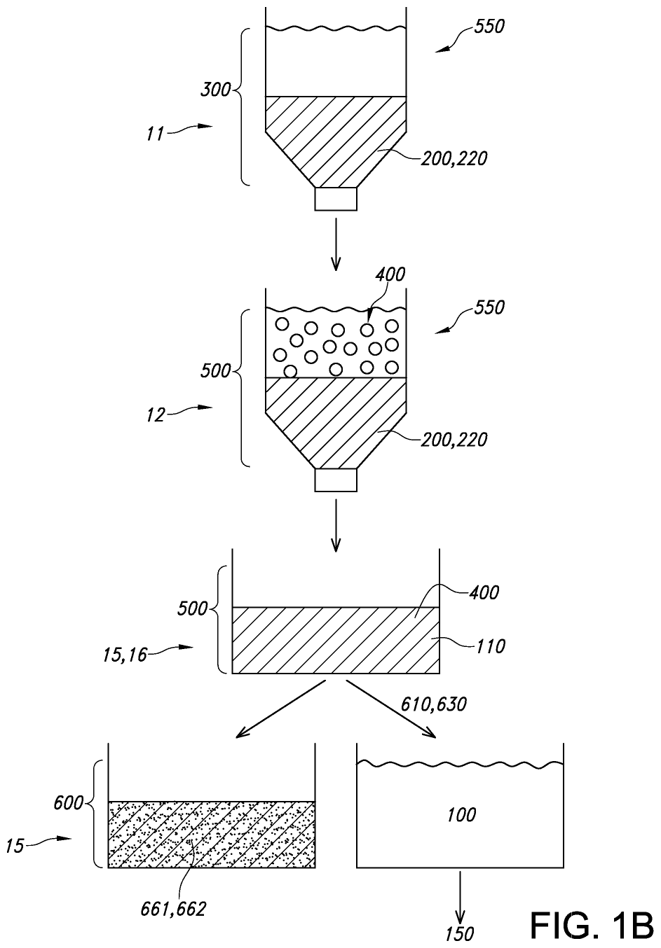

FIG. 1A-B schematically depict embodiments of a method for producing an oil composition 100 from biomass 200. In the depicted embodiments, the method comprises a preparation stage 11, a fermentation stage 12, and an oil procurement stage 15. The preparation stage 11 comprises providing a growth medium 300. The growth medium 300 comprises the biomass 200. In embodiments, the growth medium may comprise 1-10 wt % dry matter, 0.02-30 wt % of a first carbon source, 0.01-5 wt % of a first nitrogen source, and 0.00001-0.05 wt % of a magnesium source. In embodiments, the biomass 200 may comprise non-pasteurized biomass 220. The growth medium 300 further has a pH selected from the range of 3.0-8.0. The fermentation stage 12 comprises inoculating the growth medium 300 with a yeast cell 400, thereby providing a cell suspension 500. The fermentation stage further comprises controlling a temperature of the cell suspension 500 in the range of 10-40° C. The yeast cell 400 is especially selected from the species Yarrowia lipolytica or Cutaneotrichosporon oleaginous. The fermentation stage is continued until the cell suspension 500 reaches a cell density selected from the range of 1E8-1E10 cells/ml. The oil procurement stage 15 comprises separating a yeast oil 110 from the cell suspension 500 to provide the oil composition 100. This may be achieved by mechanically lysing the cell suspension 500 to provide a lysed cell suspension 600, especially wherein the mechanical lysing 610 comprises one or more of screw-pressing 630, bead beating, French pressing and homogenizing. The oil composition 100 may be processed to provide a product 150 comprising the oil composition 100.

FIG. 1A schematically depicts embodiments comprising a separation substage 17 that comprises using centrifugation 620 on the lysed cell suspension 600 to provide a supernatant 650 and a pellet 660. This may especially be the case for embodiments where the mechanical lysing comprises one or more of bead beating, French pressing, and homogenizing. The pellet may comprise cellular debris 661 and biomass residue 662. The supernatant 650 may comprise yeast oil 110 and water 651 which may be separated to provide the oil composition 100.

FIG. 1B schematically depicts embodiments comprising a drying substage 16 wherein the cell suspension 500 is dried to comprise 70-97 wt % dry weight. The drying substage is then followed by screw-pressing 630 to (i) provide the lysed cell suspension 600 comprising cellular debris 661 and biomass residue 662 and (ii) separate the oil composition 100.

The preparation stage 11 and the fermentation stage 12 may, in embodiments, occur in a (simple) fermentation vessel 550 exposed to open air, i.e., the preparation stage may comprise providing the growth medium to the fermentation vessel 550, and the fermentation stage may comprise keeping the cell suspension in the fermentation vessel 550. FIG. 1A schematically depicts embodiments where the fermentation vessel 550 comprises a fermentation tank. FIG. 1B schematically depicts embodiments where the fermentation vessel 550 comprises a fermentation barrel.

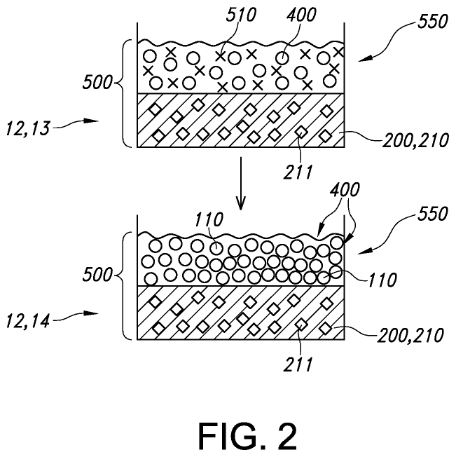

FIG. 2 schematically depicts embodiments of a method wherein the fermentation stage 12 comprises a nutrient abundance substage 13 and a nutrient limitation substage 14. During the nutrient abundance substage 13, the cell suspension 500 may comprise the yeast cell 400 and a nutrient source 510, especially a magnesium source and/or a nitrogen source. During the nutrient limitation substage 14, the cell suspension 500 comprises the yeast cell 400, but may be essentially devoid of the nutrient source, especially of at least a nitrogen source, or especially of at least a magnesium source. The yeast cell 400 may especially produce the yeast oil 110 predominantly during the nutrient limitation substage 14.

In the depicted embodiment, the biomass 200 comprises decomposed biomass 210 comprising a decomposition compound 211.

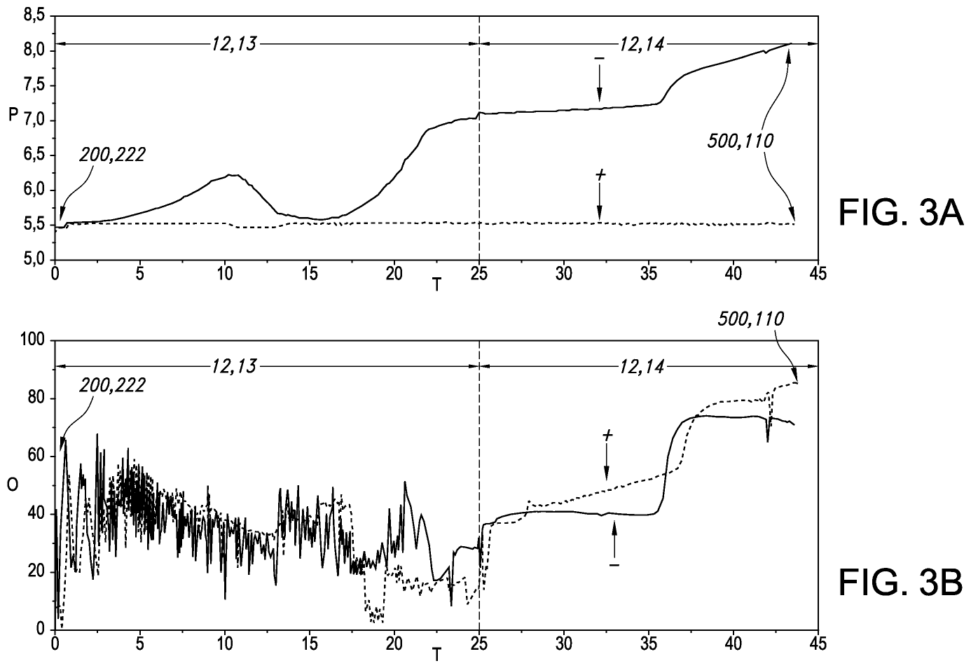

FIG. 3A-B depict experimental results obtained with embodiments of the method for producing an oil composition 100 from biomass 200. Two fermentation experiments were performed in a 2 L fermentation vessel 550 using carrot permeate water (a sidestream biomass 200 rich in sugar) as first carbon source. Especially, carrot permeate water was not sterilized or pasteurized, thereby providing a non-heated biomass 222 as first carbon source. Cutaneotrichosporon oleaginous was used as the yeast cell 400. The pH value of the growth medium 300 was set to pH 5.5 during the preparation stage 11. The temperature of the cell suspension 500 during the fermentation stage 12 was controlled at the range of 27.5-30° C. The oxygen saturation (or: “dissolved oxygen value”) in the cell suspension 500 was controlled during the nutrient abundance substage 13 (herein comprising the first 25 hours) by controlling an aeration of the cell suspension 500 using a stirring apparatus. The air saturation in the cell suspension 500 was not controlled during the nutrient limitation substage 14 (i.e., herein the air saturation was not controlled after the first 25 hours) during the yeast oil production phase (or: “yeast oil accumulation phase”). Thereby, after the fermentation stage 12, a cell suspension 500 comprising yeast oil 110 was provided. Afterwards, the yeast oil 110 was (i) quantified by gravimetric evaluation using heptane and (ii) characterized in terms of fatty acid profile by heptane extraction followed by gas chromatography.

Reference “+” indicates the results from a first fermentation experiment. Herein, the pH was controlled during the fermentation stage 12 at pH 5.5. Further, nutrients such as yeast extract, MgSO4.7H2O, and KH2PO4 were added in the concentration of 1 g/L, 0.1 g/L, and 0.1 g/L, respectively. Reference “-” indicates the results from the second fermentation experiment. Herein, the pH was not controlled during the fermentation stage 12.

FIG. 3A depicts a graph showing the pH value (“P”) of the cell suspension 500 on the vertical axis and the time (T) in hours of the fermentation stage 12 on the horizontal axis. FIG. 3B depicts a graph showing the dissolved oxygen value pO2 (O) of the cell suspension 500 on the vertical axis and the time (T) in hours of the fermentation stage 12 on the horizontal axis.

The amount of yeast oil 110 in the cell suspension 500 was determined.

| Experiment | Total amount of yeast oil 110 |
|---|---|
| + | 4.55 g |
| − | 3.70 g |

The fatty acid profile of yeast oil 110 in the cell suspension 500 was determined.

| | Fatty acid profile of yeast oil 110 | | | | | | | | | | | |
|---|---|---|---|---|---|---|---|---|---|---|---|---|
| xperiment | SFA | MUFA | PUFA | C12 | C14 | C15 | C16 | C17 | C18 | C20 | C22 | C24 |
| + | 35.8 | 53.7 | 10.4 | 0.0 | 0.3 | 0.1 | 13.8 | 0.1 | 81.4 | 1.1 | 0.6 | 2.5 |
| − | 35.2 | 53.0 | 11.1 | 0.0 | 0.3 | 0.0 | 11.2 | 0.1 | 82.0 | 1.3 | 0.7 | 2.4 |

The total amount of yeast oil 110 and fatty acid profile of both fermentation experiments are similar. Therefore, these results demonstrate that is possible to achieve the same oil quality without controlling the pH value. The pH controlled condition didn't result in the synthesis of substantially more yeast oil 110 or yeast oil 110 having substantially different fatty acid profile. The conditions used in the experiments (no pH control during the fermentation stage 12, no addition of nutrients during the fermentation stage 12, and direct fermentation of non-heated biomass 222) demonstrate that the method herein may require no specialized fermentation equipment and may be applicable to a wide variety of biomass sources, hence it may be an easily available and economically feasible method to provide safe and ethical oil compositions with desired characteristics.

The term “plurality” refers to two or more. Furthermore, the terms “a plurality of” and “a number of” may be used interchangeably.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

The term “further embodiment” and similar terms may refer to an embodiment comprising the features of the previously discussed embodiment, but may also refer to an alternative embodiment.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method, respectively.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A method for producing an oil composition from biomass, wherein the method comprises:
a preparation stage comprising providing a growth medium, wherein the growth medium comprises the biomass, wherein the growth medium comprises 1-10 wt % dry matter, 0.02-30 wt % of a carbon source, 0.01-5 wt % of a nitrogen source, 0.00001-0.05 wt. % of a magnesium source, and wherein the growth medium has a pH selected from the range of 3.0-8.0, wherein the biomass comprises a non-pasteurized biomass;
a fermentation stage comprising (a) inoculating the growth medium with a yeast cell, thereby providing a cell suspension, and (b) controlling a temperature of the cell suspension in the range of 10-40° C., wherein the yeast cell is selected from the species Yarrowia lipolytica and Cutaneotrichosporon oleaginous, and wherein the fermentation stage is continued until the cell suspension reaches a cell density selected from the range of 1E8-1E10 cells/ml, wherein the pH of the cell suspension is not controlled during the fermentation stage;
an oil procurement stage comprising separating a yeast oil from the cell suspension to provide the oil composition, wherein the oil procurement stage comprises mechanically lysing the cell suspension to provide a lysed cell suspension, wherein the mechanical lysing comprises one or more of screw-pressing, bead beating, French pressing or homogenizing.

2. The method according to claim 1, wherein the biomass comprises one or more of a side stream, a waste stream, or a residual stream from the food industry and/or agricultural industry.

3. The method according to claim 1, wherein the biomass comprises decomposed biomass comprising 0.01-10 wt % of a decomposition compound, wherein the decomposition compound comprises at least one or more of ethanol, acetic acid, or lactic acid.

4. The method according to claim 1, wherein the fermentation stage has a duration selected from the range of 2-20 days, and wherein the fermentation stage comprises periodically adding 0.05-5 wt % of a second carbon source to the cell suspension at an interval selected from the range of 62-48 hours.

5. The method according to claim 1, wherein the fermentation stage comprises controlling a temperature of the cell suspension in the range of 10-24° C.

6. The method according to claim 1, wherein the fermentation stage comprises controlling a temperature of the cell suspension in the range of 25-40° C.

7. The method according to claim 1, wherein the fermentation stage comprises controlling an aeration in the cell suspension exposed to open air to provide 5-30% air saturation in the cell suspension.

8. The method according to claim 1, wherein the fermentation stage is executed in a fermentation vessel, wherein the fermentation vessel is a fermentation tank, a fermentation vat, or a fermentation barrel; wherein the fermentation stage does not comprise monitoring of air saturation or PH level of the cell suspension.

9. The method according to claim 1, wherein the fermentation stage comprises a nutrient limitation substage, wherein during the nutrient limitation substage the cell suspension comprises 0.001-0.5 wt %. of nitrogen source, 0.000001-0.01 wt %. of bioavailable Mg²⁺ magnesium source, and 0.05-20 wt % of carbon source; and wherein the nutrient limitation substage has a nutrient limitation duration of at least 2 days.

10. The method according to claim 1, wherein the fermentation stage is continued until the cell suspension comprises at least 30 wt % of yeast oil.

11. The method according to claim 1, wherein the oil procurement stage comprises a drying substage comprising drying the cell suspension to at most 30 wt % liquid content, and wherein the mechanical lysing comprises screw-pressing.

12. The method according to claim 1, wherein the mechanical lysing comprises one or more of bead beating, French pressing, or homogenizing; and wherein the oil procurement stage further comprises a separation substage comprising centrifuging the lysed cell suspension to provide a supernatant and a pellet, and wherein the separation substage comprises separating the yeast oil from a water phase in the supernatant to provide the oil composition.

13. The method according to claim 1, wherein the yeast cell is selected from the species Yarrowia lipolytica; and wherein the mechanical lysing comprises one or more of screw-pressing, bead beating, or French pressing.

14. The method according to claim 1, wherein the yeast cell is selected from the species Cutaneotrichosporon oleaginous; and wherein the mechanical lysing comprises screw-pressing or French pressing.

15. The method according to claim 1, wherein the biomass comprises a non-heated biomass, wherein the growth medium has a pH lower than 5.0, wherein no nutrients are added during the fermentation stage, and wherein the fermentation stage does not comprise monitoring of oxygen saturation or pH level of the cell suspension.

16. The method according to claim 15, wherein:
the biomass comprises one or more of a side stream, a waste stream, or a residual stream from the food industry and/or agricultural industry;
the biomass comprises decomposed biomass comprising 0.01-10 wt % of a decomposition compound, wherein the decomposition compound comprises at least one or more of ethanol, acetic acid, or lactic acid; and
the fermentation stage is executed in a fermentation vessel, wherein the fermentation vessel is a fermentation tank, a fermentation vat, or a fermentation barrel.

17. The method according to claim 15, wherein, relative to a total volume of the growth medium at least 99 vol. % of the water content of the growth medium is provided by the biomass, and wherein, relative to a total volume of the cell suspension, at most 1 vol. % water is added to the cell suspension during the fermentation stage.

18. The method according to claim 15, wherein the oxygen saturation of the cell suspension is not controlled during the fermentation stage.

19. The method according to claim 15, wherein the oxygen saturation of the cell suspension is controlled during the nutrient abundance substage by controlling an aeration of the cell suspension, and wherein the oxygen saturation of the cell suspension is not controlled during the nutrient limitation substage.

20. (canceled)

21. A product comprising the oil composition obtainable by the method according to claim 1, wherein the product includes food products, cosmetic products, cleaning products, fuels, and medical products.
